(19) United States
(12) Patent Application Publication
(10) Pub. No.: US 2024/0254160 A1
(43) Pub. Date: Aug. 1, 2024

(54) SEPARATION OF PRE-PEAK AND POST-PEAK IN FUSION PROTEIN SAMPLE BY USING SIZE EXCLUSION HIGH PERFORMANCE LIQUID CHROMATOGRAPHY

(71) Applicant: Kashiv BioSciences, LLC, Piscataway, NJ (US)

(72) Inventors: Roshan Ganeshlal Upadhyay, Ahmedabad (IN); Darshana Jiten Maniar, Ahmedabad (IN); Shivani Singh, Ahmedabad (IN)

(21) Appl. No.: 18/629,465

(22) Filed: Apr. 8, 2024

Related U.S. Application Data
(63) Continuation of application No. PCT/IB2022/059649, filed on Oct. 8, 2022.

(30) Foreign Application Priority Data
Oct. 8, 2021 (IN) 202121045959

(51) Int. Cl. C07K 1/16; B01D 15/34; B01J 20/28; B01J 20/283; C07K 14/705

(52) U.S. Cl. CPC C07K 1/16; B01D 15/34; B01J 20/28083; B01J 20/283; C07K 14/70521; C07K 2319/30

(57) ABSTRACT

The present invention provides an effective High Performance Liquid Chromatography (SE-HPLC) method to separate or resolve the pre-peak, post-peak and main peak of CTLA4-IgG1 fusion protein, where pre-peak resolution is more than 1.3. The invention further provides effective separation and resolution of post peak impurity present in CTLA4 IgG1 protein. Moreover, the present invention also provides the method for the estimation and/or quantification of pre-peak, post-peak and main peak of the protein mixture.

### FIELD OF THE INVENTION

The present invention provides an effective High Performance Liquid Chromatography (SE-HPLC) method to separate or resolve the pre-peak, post-peak and main peak of CTLA4-IgG1 fusion protein, where pre-peak resolution is more than 1.3. The invention further provides effective separation and resolution of post peak impurity present in CTLA4 IgG1 protein. Moreover, the present invention also provides the method for the estimation and/or quantification of pre-peak, post-peak and main peak of the protein mixture.

### BACKGROUND OF THE INVENTION

In the production of biologics, it is very important to develop robust process to provide protein with high purity and less impurities especially high molecular weight impurities (HMWs) and low molecular weight impurities (LMWs). In order to establish a successful downstream process, it is very imperative to analyze the post-harvest protein mixture to evaluate or characterize the impurities such as HMWs and LMWs. Size exclusion High Performance Liquid Chromatography is a technique to estimate or quantify pre-peak and post-peak but resolving a pre-peak and post-peak from main peak is very challenging and it is observed that routine Size exclusion High Performance Liquid Chromatography does not provide sharp resolution of pre-peak, post-peak and main peak of complex proteins such as antibody or fusion proteins. In absence of obtaining a sharp resolution, it is very difficult for skilled person to quantify the presence of pre-peak and post-peak adequately and it further creates uncertainty about the impurities during down-stream purification (DSP) which makes the DSP process expensive, ineffective, and lengthy. Therefore, it is very important to have an effective, robust Size exclusion High Performance Liquid Chromatography process to separate, estimate or quantify impurities such as HMW and LMW.

The present invention solves the problem and provide effective, robust Size exclusion High Performance Liquid Chromatography process to separate, estimate and/or quantify impurities present in fusion protein mixture such as high molecular weight impurities (HMWs) and low molecular weight impurities (LMWs).

### SUMMARY OF THE INVENTION

In an embodiment, the invention provides the process for performing Size exclusion High Performance Liquid Chromatography to estimate and/or quantify impurities such as HMWs and LMWs present in protein mixture.

In an embodiment the method for the separation of CTLA4-IgG1 fusion protein mixture comprising CTLA4-IgG1 fusion protein, pre-peak and post-peak impurity thereof, the process comprises;
a) loading the CTLA4-IgG1 fusion protein mixture onto Size exclusion High Performance Liquid Chromatography (SE-HPLC) column;
b) separating the protein mixture with suitable mobile phase comprising combination of salts at suitable pH more than pI of the fusion protein; wherein the mobile phase maintains flow rate more than 0.3 mL/min;
c) separating the pre-peak impurity and post-peak impurity from the CTLA4-IgG1 fusion protein of interest, wherein the separation has pre-peak resolution is more than 1.3.

In an embodiment, the present invention provides a method for the separation of CTLA4-IgG1 fusion protein mixture comprising CTLA4-IgG1 fusion protein, pre-peak and post-peak impurity thereof, the process comprising;
a) loading the CTLA4-IgG1 fusion protein mixture onto Size exclusion High Performance Liquid Chromatography (SE-HPLC) column;
b) separating the protein mixture with suitable mobile phase comprising combination of salts at suitable pH more than pI of the fusion protein; wherein the mobile phase maintains flow rate more than 0.3 mL/min;
c) separating the pre-peak impurity and post-peak impurity from fusion protein of interest, wherein the separation has pre-peak resolution is more than 1.3.

In an embodiment, the invention separates the pre-peak, post-peak and main peak of fusion protein at suitable flow rate above 0.3 ml/min.

In an embodiment, the invention separates the pre-peak, post-peak and main peak of fusion protein at suitable flow rate selected from 0.35 ml/min, 0.4 ml/min, about 0.45 ml/min, about 0.5 ml/min, about 0.55 ml/min, about 0.6 ml/min, about 0.65 ml/min about 0.7 ml/min, about 0.75 ml/min and about 0.8 ml/min.

In an embodiment, the SE-HPLC column has silica matrix and pore size is selected from about 14 nm or 140 Å to about 29 nm or 290 Å.

In an embodiment, the SE-HPLC column has silica matrix and pore size is 14.5 nm or 145 Å.

In an embodiment, the loading concentration of protein mixture is selected from about 0.5 mg/ml to about 1.4 mg/ml.

In an embodiment, the loading amount of protein mixture is selected from about 10 μg to about 100 μg.

In an embodiment, the protein mixture can be obtained selected from cell culture harvest, protein A eluate, mixed mode chromatography eluate, anion exchange chromatography eluate, cation exchange chromatography eluate or after any other purification steps.

In an embodiment, the protein mixture can be obtained from harvest, partially purified, substantially purified by any other purification methods.

In an embodiment, the protein mixture is obtained from affinity chromatography, preferably protein A chromatography.

In an embodiment, the SE-HPLC column comprising silica-based resin. In certain embodiment, the column pore size is more than 120 Å. In an embodiment, the column pore size is selected from 14.5 nm or 145 Å to 29 nm or 290 Å. In certain embodiment, the columns are selected from Biosep SEC s2000, Biosep SEC s3000, Biosep SEC s4000.

In an embodiment, the invention provides USP peak tailing is from about 0.7 to about 1.15.

In certain embodiment, the pre-peak and post-peaks are not merged or interfered with main peak.

In an embodiment, the suitable detection absorbance is selected from about 214 nm to about 280 nm. In an embodiment, the detection absorbance is 215 nm.

In an embodiment, the invention provides purity of fusion protein of interest or main peak more than 98%.

In an embodiment, the present invention provides an improved method for quantification and/or estimation of impurities in a protein sample comprising;
a) a protein mixture from harvest comprising protein of interest and size variant impurities;
b) loading the protein mixture to said Size exclusion High Performance Liquid Chromatography (SE-HPLC) column;
c) separating the protein mixture with suitable mobile phase comprising combination of salts at suitable pH;
d) analysed or quantified the pre-peak, post-peak and main peak of the protein mixture at suitable detection absorbance.

In an embodiment, the suitable pH of mobile phase is selected from about 5.5 to about 7.0.

In certain embodiment, the suitable pH of mobile phase is selected from about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0.

In an embodiment the method for the separation of CTLA4-IgG1 fusion protein mixture comprising CTLA4-IgG1 fusion protein, pre-peak and post-peak impurity thereof, the process comprises;
a) loading the CTLA4-IgG1 fusion protein mixture onto Size exclusion High Performance Liquid Chromatography (SE-HPLC) Biosep SEC s2000 column;
b) separating the protein mixture with suitable mobile phase comprising combination of salts at suitable pH; wherein the salts is selected from phosphate, sodium and sulphate salts. wherein the mobile phase maintains flow rate more than 0.3 mL/min.
c) separating the pre-peak impurity and post-peak impurity from the CTLA4-IgG1 fusion protein of interest, wherein the separation has pre-peak resolution is more than 1.3.
d) analysed or quantified the pre-peak, post-peak and main peak of the CTLA4-IgG1 fusion protein mixture.

In certain embodiment, the pre-peak area and post-peak is not merged or interfered with main peak area.

In an embodiment, the present invention provides an improved method for quantification and/or estimation of impurities in a protein sample comprising;
a) a protein mixture from harvest comprising protein of interest and size variants impurities;
b) loading the protein mixture to said Size exclusion High Performance Liquid Chromatography (SE-HPLC) Biosep SEC s2000 column;
c) separating the protein mixture with suitable mobile phase comprising combination of salts at suitable pH; wherein the salts are Sodium phosphate in combination with Sodium sulphate in suitable concentration;
d) analysed or quantified the pre-peak, post-peak and main peak of the protein mixture; wherein the SE-HPLC column provides the resolution of pre-peak is more than 1.3.

In such embodiment, the concentration of mobile phase Sodium phosphate is selected from about 50 mM, about 60 mM, about 65 mM, about 70 mM, about 75 mM, about 80 mM, about 85 mM, about 90 mM, about 95 mM, about 100 mM, about 105 mM, about 110 mM, 115 mM, about 120 mM, about 125 mM, about 130 mM, about 135 mM, about 140 mM, about 145 mM, and about 150 mM.

In such embodiment, the concentration of mobile phase Sodium sulphate is selected from about 50 mM, about 60 mM, about 65 mM, about 70 mM, about 75 mM, about 80 mM, about 85 mM, about 90 mM, about 95 mM, about 100 mM, about 105 mM, about 110 mM, 115 mM, about 120 mM, about 125 mM, about 130 mM, about 135 mM, about 140 mM, about 145 mM, and about 150 mM.

In an embodiment, the present invention provides an improved method for quantification and/or estimation of impurities in a protein sample comprising;
a) a protein mixture from harvest comprising protein of interest and size variant impurities;
b) loading the protein mixture to said Size exclusion High Performance Liquid Chromatography (SE-HPLC) Biosep SEC s2000 column;
c) separating the protein mixture with suitable mobile phase comprising combination of salts at suitable pH; wherein the salts are 100 mM Sodium phosphate in combination with 100 mM Sodium sulphate in suitable concentration;
d) analysed or quantified the pre-peak, post-peak and main peak of the protein mixture wherein the SE-HPLC column provides the resolution of pre-peak is more than 1.3.

In an embodiment, the present invention provides an improved method for quantification and/or estimation of impurities in a protein sample comprising;
a) a protein mixture from harvest comprising protein of interest and size variant impurities;
b) loading the protein mixture to said Size exclusion High Performance Liquid Chromatography (SE-HPLC) Biosep SEC s2000 column;
c) separating the protein mixture with suitable mobile phase comprising combination of salts at suitable pH; wherein the salts are 100 mM Sodium phosphate in combination with 200 mM Sodium sulphate in suitable concentration;
d) analysed or quantified the pre-peak, post-peak and main peak of the protein mixture wherein the SE-HPLC column provides the resolution of pre-peak is more than 1.3.

In such embodiment, the concentration of mobile phase Sodium sulphate is selected from about 50 mM, about 60 mM, about 65 mM, about 70 mM, about 75 mM, about 80 mM, about 85 mM, about 90 mM, about 95 mM, about 100 mM, about 120 mM, about 130 mM, about 140 mM, about 150 mM, about 160 mM, about 170 mM, about 180 mM, about 190 mM, about 200 mM, about 210 mM, and about 220 mM.

In certain embodiment, the pH of mobile phase is selected from about 5.5, about 6.0, about 6.1, about 6.2, about 6.3, about 6.4, about 6.5, about 6.6, about 6.7, about 6.8, about 6.9, and about 7.0.

In an embodiment, the pH of mobile phase is 6.5.

In an embodiment, the present invention provides an improved method for quantification and/or estimation of impurities in a protein sample comprising;
a) a protein mixture from harvest comprising CTLA4-IgG1 and size variant impurities;
b) loading the protein mixture to said Size exclusion High Performance Liquid Chromatography (SE-HPLC) Biosep SEC s2000 column;
c) separating the protein mixture with suitable mobile phase comprising combination of 100 mM Sodium phosphate in combination with 100 mM Sodium sulphate at pH 6.5;
d) analysed or quantified the pre-peak, post-peak and main peak of the protein mixture wherein the SE-HPLC column provides the resolution of pre-peak is more than 1.3.

In an embodiment, the present invention provides an improved method for quantification and/or estimation of impurities in a protein sample comprising;
a) a protein mixture from harvest comprising CTLA4-IgG1 and size variant impurities;
b) loading the protein mixture to said Size exclusion High Performance Liquid Chromatography (SE-HPLC) Biosep SEC s2000 column;
c) separating the protein mixture with suitable mobile phase comprising combination of 100 mM Sodium phosphate in combination with 200 mM Sodium sulphate at pH 6.5
d) analysed or quantified the pre-peak, post-peak and main peak of the protein mixture; wherein the SE-HPLC column provides the resolution of pre-peak is more than 1.3.

In an embodiment, the resolution of pre-peak is from about 1.3 to about 1.9.

### BRIEF DESCRIPTION OF FIGURES

FIG. 1 shows separation of post-peak in Biosep SEC s2000 column.

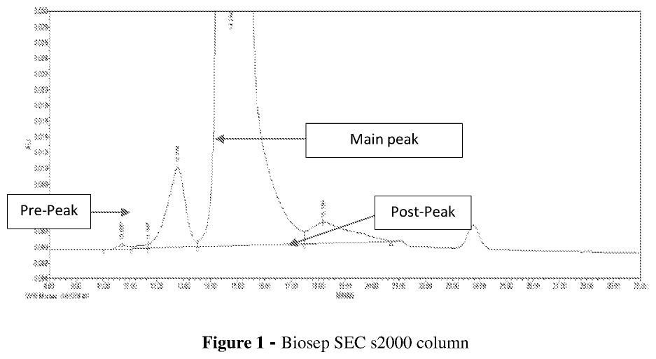

FIG. 2 shows no separation of post-peak in TSK gel G3000swxl column.

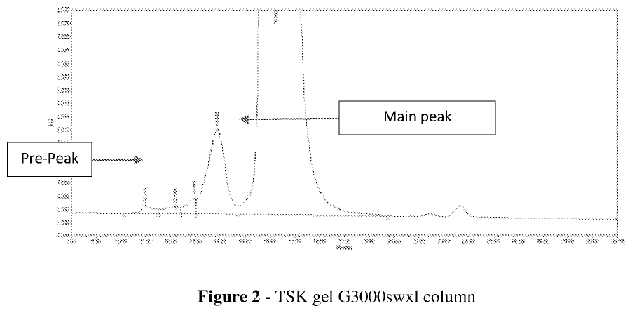

FIG. 3 shows the comparative effect of mobile phase having 100 mM Sodium phosphate with 100 mM Na₂SO₄, 100 mM and 200 mM NaCl, pH 6.5.

FIG. 4 shows the comparative effect of mobile phase having 100 mM Sodium phosphate with 100 mM Na₂SO₄ compared with mobile phase having Potassium phosphate with 200 mM KCl, pH 6.5.

FIG. 5 shows the results Sample (Reference CTLA4-IgG1) with 0.5 ml/min flow rate.

FIG. 6 shows the results Sample (Reference CTLA4-IgG1) with 0.3 ml/min flow rate.

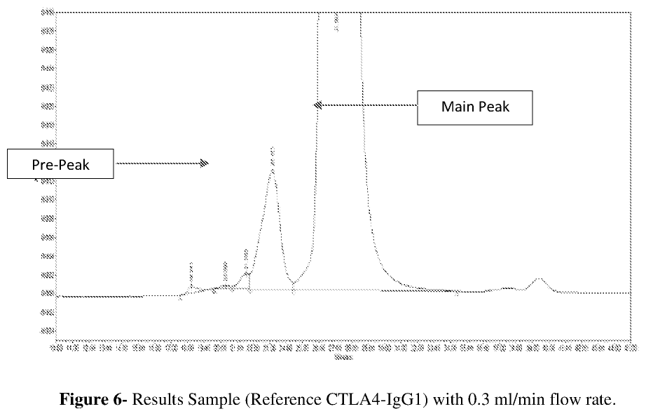

### DETAIL DESCRIPTION OF THE INVENTION

The present invention relates to an improved method for analysis of protein mixture comprises of at least one antibody or fusion protein, wherein the analysis of protein mixtures is performed with Size Exclusion High Performance Liquid Chromatography (SE-HPLC).

The term “Size Exclusion High Performance Liquid Chromatography (SE-HPLC)” refers to chromatography processes that employs porous particles in the column to separate molecules by virtue of their size in solution. SE-HPLC is generally used to separate biological molecules, to determine molecular weight distributions of proteins. Hence size variants of the protein CTAL4-IgG1 or CTLA4-IgG1 fusion protein can be separated by SE HPLC and purity of the main peak of CTLA4-IgG1 fusion protein can be determined. The separation can be achieved by using size exclusion column with isocratic elution using a mobile phase and detection by UV at 215 nm.

As used throughout the specification and in the appended claims, the singular forms “a”, “an”, and “the” include the plural reference unless the context clearly dictates otherwise.

The term “about”, as used herein, is intended to refer to ranges of approximately 10-20% greater than or less than the referenced value. In certain circumstances, one of skill in the art will recognize that, due to the nature of the referenced value, the term “about” can mean more or less than a 10-20% deviation from that value.

The term “comprises” or “comprising” is used in the present description, it does not exclude other elements or steps. For the purpose of the present invention, the term “consisting of” is considered to be an optional embodiment of the term “comprising of”. If hereinafter a group is defined to comprise at least a certain number of embodiments, this is also to be understood to disclose a group which optionally consists only of these embodiments.

The term “CTLA4-IgG1” or “CTLA4-IgG1 fusion protein” or “fusion protein of interest” or “fusion protein” used herein are interchangeable refers to a recombinant DNA generated fusion protein used to treat the symptoms of rheumatoid arthritis and to prevent joint damage caused by these conditions. CTLA4-IgG1 fusion protein is a biological product developed for immunosuppression by blocking T cell activation through inhibition of costimulatory signals and is indicated for treatment of rheumatoid arthritis. CTLA4-IgG1 fusion protein is a soluble homodimeric fusion protein of two identical subunits covalently linked by one disulfide bond. Each subunit consists of the modified amino acid sequence of the human cytotoxic lymphocyte associated antigen 4 (CTLA4), human immunoglobin IgG1 hinge, CH2 and CH3 region (Fc). Modification to the original sequences were introduced to avoid unintended disulfide bond formation and to reduce the ability of complement activation. Fusion protein examples such as TNF receptor 2-Fc (etanercept), rilonacept (Arcalyst—an IL-1 Trap), vascular endothelial growth factor trap (aflibercept), CTLA4-Fc fusion proteins (Abatacept and belatacept).

The term “protein mixture” and “protein sample” are interchangeable respectively in the present invention.

The term “Percentage (%) purity” refers to the percent of purity that determine the purity of protein present in the sample.

The term used “Percentage (%) purity” and “main peak area percentage (%)” are interchangeable respectively in the present invention.

The term “Percentage (%) molecular weight related impurities” refers to percent of high molecular weight impurities and low molecular weight impurities.

The term “pre peak area percentage (%)” refers to the percent of peak area that comes before the main peak area. The pre peak area includes high molecular weight aggregates.

The term “post peak area percentage (%)” refers to the percent of peak area that comes after the main peak area. The post peak area includes low molecular weight aggregates.

The term used “high molecular weight” or “HMW” is product-related impurities that contribute to the size heterogeneity of fusion protein drug product. The formation of HMW species within a therapeutic fusion protein drug product as a result of protein aggregation can potentially compromise both drug efficacy and safety (e.g., eliciting unwanted immunogenic response). HMW is considered critical quality attribute that are routinely monitored during drug development and as part of release testing of purified drug product during manufacturing. In certain embodiment the HMW relates to aggregates.

The term used “low molecular weight” or “LMW” species which is a protein backbone-truncated fragments & considered as product-related impurities that contribute to the size heterogeneity of fusion protein. LMW species often have low or substantially reduced activity relative to the monomeric form of the fusion protein and can lead to immunogenicity or potentially impact pharmacokinetic properties in vivo. As a result, LMW species are considered critical quality attributes that are routinely monitored during drug development and as part of release testing of purified drug product during manufacturing.

The term “column” refers to the column of SE-HPLC selected from bioZen SEC-2, bioZen SEC-3, MabPac SEC-1, BioBasic SEC 60, BioBasic SEC 120, YMC SEC Mab, YMC-Pack Diol-200, Biosep SEC s4000, Biosep SEC s3000 and Biosep SEC s2000.

In an embodiment, the column used for SE-HPLC selected from MabPac SEC-1, YMC SEC Mab, Biosep SEC s3000 and Biosep SEC s2000.

In preferred embodiment, the column used for SE-HPLC is Biosep SEC s2000.

The term “pI” or “Isoelectric point” used herein are interchangeable refers to the pH of a solution at which the net charge of a protein becomes zero. At solution pH that is above the pI, the surface of the protein is predominantly negatively charged, and therefore like-charged molecules will exhibit repulsive forces. Likewise, at a solution pH that is below the pI, the surface of the protein is predominantly positively charged, and repulsion between proteins occurs. The pI of CTLA4-IgG1 is less than 6.5.

The term “mobile phase” refers to mobile phase having salts selected from sodium phosphate, sodium sulphate, sodium chloride, potassium phosphate, potassium chloride, calcium chloride, and calcium phosphate.

The term “flow rate” refers to amount of mobile phase passing through the column in unit time.

In an embodiment, mobile phase having salts selected from sodium phosphate, sodium sulphate, potassium phosphate, and potassium chloride.

In other embodiment, the mobile phase having salts selected from sodium phosphate in combination with sodium sulphate, potassium phosphate in combination with potassium chloride, sodium phosphate in combination with potassium chloride, and potassium phosphate in combination with sodium sulphate.

In preferred embodiment, mobile phase having salts are sodium phosphate in combination with sodium sulphate.

In an embodiment, the present invention provides a process for the separation of protein mixture comprising fusion protein of interest, pre-peak impurity, and post-peak impurity, the process comprising;
a) loading the protein mixture onto Size exclusion High Performance Liquid Chromatography (SE-HPLC) column;
b) separating the protein mixture with suitable mobile phase comprising combination of salts at suitable pH more than pI of the fusion protein; wherein the mobile phase maintains flow rate more than 0.3 mL/min;
c) separating the pre-peak impurity and post-peak impurity from fusion protein of interest, wherein the separation has pre-peak resolution is more than 1.3.

In an embodiment, the invention separates the pre-peak, post-peak and main peak of fusion protein at suitable flow rate above 0.3 ml/min.

In an embodiment, the invention separates the pre-peak, post-peak and main peak of fusion protein at suitable flow rate selected from 0.35 ml/min, 0.4 ml/min, about 0.45 ml/min, about 0.5 ml/min, about 0.55 ml/min, about 0.6 ml/min, about 0.65 ml/min about 0.7 ml/min, about 0.75 ml/min and about 0.8 ml/min.

In an embodiment, the loading concentration of protein mixture is selected from about 0.5 mg/ml to about 1.4 mg/ml.

In an embodiment, the loading amount of protein mixture is selected from about 10 μg to about 100 μg.

In an embodiment, the loading of protein mixture comprises about 30 μg/μl to about 80 μg/μl.

In an embodiment, the loading of protein mixture is about 30 μg/μl.

In an embodiment, the protein mixture can be obtained selected from cell culture harvest, protein A eluate, mixed mode chromatography eluate, anion exchange chromatography eluate, cation exchange chromatography eluate or after any other purification steps.

In an embodiment, the SE-HPLC column comprising silica-based resin preferably diol type silica-based resin. In certain embodiment, the column pore size is more than 120 Å. In an embodiment, the column pore size is selected from 14.5 nm or 145 Å to 50 nm or 500 Å. In certain embodiment, the columns are selected from Biosep SEC s2000, Biosep SEC s3000, Biosep SEC s4000.

In an embodiment, the invention provides USP peak tailing is from about 0.7 to about 1.15.

In certain embodiment, the pre-peak and post-peaks are not merged or interfered with main peak.

In an embodiment, the suitable detection absorbance is selected from about 214 nm to about 280 nm. In an embodiment, the detection absorbance is 215 nm.

In an embodiment, the invention provides purity of fusion protein of interest or main peak more than 98%.

In an embodiment, the present invention provides an improved method for quantification and/or estimation of impurities in a protein sample comprising;
a) a protein mixture from harvest comprising protein of interest and size variant impurities;
b) loading the protein mixture to said Size exclusion High Performance Liquid Chromatography (SE-HPLC) column;
c) separating the protein mixture with suitable mobile phase comprising combination of salts at suitable pH;
d) analysed or quantified the pre-peak, post-peak and main peak of the protein mixture at suitable detection absorbance.

In an embodiment, the suitable pH of mobile phase is selected from about 5.5 to about 7.0.

In certain embodiment, the suitable pH of mobile phase is selected from about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0.

In an embodiment, the present invention provides an improved method for quantification and/or estimation of impurities in a protein sample comprising;
a) a protein mixture from harvest comprising protein of interest and size variant impurities;
b) loading the protein mixture to said Size exclusion High Performance Liquid Chromatography (SE-HPLC) Biosep SEC s2000 column;
c) separating the protein mixture with suitable mobile phase comprising combination of salts at suitable pH; wherein the salts is selected from phosphate, sodium and sulphate salts;
d) analysed or quantified the pre-peak, post-peak and main peak of the protein mixture.

In certain embodiment, the pre-peak area and post-peak is not merged or interfered with main peak area.

In an embodiment, the present invention provides an improved method for quantification and/or estimation of impurities in a protein sample comprising;
a) a protein mixture from harvest comprising protein of interest and size variants impurities;
b) loading the protein mixture to said Size exclusion High Performance Liquid Chromatography (SE-HPLC) Biosep SEC s2000 column;
c) separating the protein mixture with suitable mobile phase comprising combination of salts at suitable pH; wherein the salts are Sodium phosphate in combination with Sodium sulphate in suitable concentration;
d) analysed or quantified the pre-peak, post-peak and main peak of the protein mixture; wherein the SE-HPLC column provides the resolution of pre-peak is more than 1.3.

In such embodiment, the concentration of mobile phase Sodium phosphate is selected from about 60 mM, about 65 mM, about 70 mM, about 75 mM, about 80 mM, about 85 mM, about 90 mM, about 95 mM, about 100 mM, about 105 mM, about 110 mM, 115 mM, about 120 mM, about 125 mM, about 130 mM, about 135 mM, about 140 mM, about 145 mM, and about 150 mM.

In such embodiment, the concentration of mobile phase Sodium sulphate is selected from about 60 mM, about 65 mM, about 70 mM, about 75 mM, about 80 mM, about 85 mM, about 90 mM, about 95 mM, about 100 mM, about 105 mM, about 110 mM, 115 mM, about 120 mM, about 125 mM, about 130 mM, about 135 mM, about 140 mM, about 145 mM, and about 150 mM.

In an embodiment, the present invention provides an improved method for quantification and/or estimation of impurities in a protein sample comprising;
a) a protein mixture from harvest comprising protein of interest and size variant impurities;
b) loading the protein mixture to said Size exclusion High Performance Liquid Chromatography (SE-HPLC) Biosep SEC s2000 column;
c) separating the protein mixture with suitable mobile phase comprising combination of salts at suitable pH; wherein the salts are 100 mM Sodium phosphate in combination with 100 mM Sodium sulphate in suitable concentration;
d) analysed or quantified the pre-peak, post-peak and main peak of the protein mixture; wherein the SE-HPLC column provides the resolution of pre-peak is more than 1.3.

In an embodiment, the present invention provides an improved method for quantification and/or estimation of impurities in a protein sample comprising;
a) a protein mixture from harvest comprising protein of interest and size variants impurities;
b) loading the protein mixture to said Size exclusion High Performance Liquid Chromatography (SE-HPLC) Biosep SEC s2000 column;
c) separating the protein mixture with suitable mobile phase comprising combination of salts at suitable pH; wherein the salts are 100 mM Sodium phosphate in combination with 200 mM Sodium sulphate in suitable concentration;
d) analysed or quantified the pre-peak, post-peak and main peak of the protein mixture; wherein the SE-HPLC column provides the resolution of pre-peak is more than 1.3.

In such embodiment, the concentration of mobile phase Sodium sulphate is selected from about 60 mM, about 65 mM, about 70 mM, about 75 mM, about 80 mM, about 85 mM, about 90 mM, about 95 mM, about 100 mM, about 120 mM, about 130 mM, about 140 mM, about 150 mM, about 160 mM, about 170 mM, about 180 mM, about 190 mM, about 200 mM, about 210 mM, and about 220 mM.

In certain embodiment the pH is selected from about 5.5, about 6.0, about 6.1, about 6.2, about 6.3, about 6.4, about 6.5, about 6.6, about 6.7, about 6.8, about 6.9, and about 7.0.

In an embodiment, the present invention provides an improved method for quantification and/or estimation of impurities in a protein sample comprising;
a) a protein mixture from harvest comprising CTLA4-IgG1 and size variant impurities;
b) loading the protein mixture to said Size exclusion High Performance Liquid Chromatography (SE-HPLC) Biosep SEC s2000 column;
c) separating the protein mixture with suitable mobile phase comprising combination of 100 mM Sodium phosphate in combination with 100 mM Sodium sulphate at pH 6.5;
d) analysed or quantified the pre-peak, post-peak and main peak of the protein mixture; wherein the SE-HPLC column provides the resolution of pre-peak is more than 1.3.

In an embodiment, the pre-peak impurity is high molecular weight and/or aggregates and post-peak impurity is low molecular weight and/or fragments of CTLA4-IgG1.

In an embodiment, the present invention provides an improved method for quantification and/or estimation of impurities in a protein sample comprising;
a) a protein mixture from harvest comprising CTLA4-IgG1 and size variant impurities;
b) loading the protein mixture to said Size exclusion High Performance Liquid Chromatography (SE-HPLC) Biosep SEC s2000 column;
c) separating the protein mixture with suitable mobile phase comprising combination of 100 mM Sodium phosphate in combination with 200 mM Sodium sulphate at pH 6.5;
d) analysed or quantified the pre-peak, post-peak and main peak of the protein mixture; wherein the SE-HPLC column provides the resolution of pre-peak is more than 1.3.

In an embodiment, the salt concentration used in mobile phase is selected from about 50 mM, about 60 mM, about 65 mM, about 70 mM, about 75 mM, about 80 mM, about 85 mM, about 90 mM, about 95 mM, about 100 mM, about 105 mM, about 110 mM, and about 115 mM, about 120 mM, about 125 mM, about 130 mM, about 135 mM, about 140 mM, about 145 mM, and about 150 mM of Sodium phosphate.

In another embodiment, the salt concentration used in mobile phase is selected from about 80 mM, about 90 mM, about 100 mM, about 110 mM, and about 120 mM of Sodium phosphate.

In preferred embodiment, the salt concentration used in mobile phase is about 100 mM of Sodium phosphate.

In an embodiment, the salt concentration used in mobile phase is selected from about 50 mM, about 60 mM, about 65 mM, about 70 mM, about 75 mM, about 80 mM, about 85 mM, about 90 mM, about 95 mM, about 100 mM, about 105 mM, about 110 mM, and about 115 mM, 120 mM, 125 mM, 130 mM, 135 mM, 140 mM, 145 mM and about 150 mM of Sodium sulphate.

In another embodiment, the salt concentration used in mobile phase is selected from about 80 mM, about 90 mM, about 100 mM, about 110 mM, and about 120 mM of Sodium sulphate.

In preferred embodiment, the salt concentration used in mobile phase is about 100 mM of Sodium sulphate.

In an embodiment, the salt concentration used in mobile phase is selected from about 100 mM, about 120 mM, about 130 mM, about 140 mM, about 150 mM, about 160 mM, about 170 mM, about 180 mM, about 190 mM, about 200 mM, about 210 mM, and about 220 mM of Sodium sulphate.

In another embodiment, the salt concentration used in mobile phase is selected from about 100 mM, about 150 mM, and about 200 mM of Sodium sulphate.

In preferred embodiment, the salt concentration used in mobile phase is about 200 mM of Sodium sulphate.

In an embodiment, the mobile phase is free of sodium chloride, arginine, acetonitrile, TFA, guanidine hydrochloride, urea and formic acid.

In an embodiment, the pH of mobile phase is adjusted to pH selected from about pH 5.5 to about pH 7.0, about pH 6.0 to about pH 7.0, about pH 6.5 to about pH 7.0, and about pH 6.7 to about pH 7.0.

In preferred embodiment, the pH of mobile phase is adjusted to about pH 6.5.

In an embodiment, the pH of mobile phase is adjusted by acid selected from sulphuric acid, hydrochloric acid (HCI), nitric acid and phosphoric acid.

In another embodiment, the pH of mobile is adjusted by acid selected from hydrochloric acid (HCI) and phosphoric acid.

In preferred embodiment, the pH of mobile is adjusted by Orthophosphoric acid.

In an embodiment, the flow rate of mobile phase is selected from about 0.1 mL/min, about 0.2 mL/min, about 0.3 mL/min, about 0.4 mL/min, about 0.5 mL/min, about 0.6 mL/min, about 0.7 mL/min, about 0.8 mL/min, about 0.9 mL/min and about 1.0 mL/min.

In another embodiment, the flow rate of mobile phase is selected from about 0.1 mL/min, about 0.2 mL/min, about 0.3 mL/min, about 0.4 mL/min, and about 0.5 mL/min.

In an embodiment, the flow rate of mobile phase is less than 0.6 mL/min.

In preferred embodiment, the flow rate of mobile phase is about 0.5±0.2 mL/min.

In an embodiment, the mobile phase is free of sodium chloride, arginine, acetonitrile, TFA, guanidine hydrochloride, urea and formic acid.

In an embodiment, the resolution of pre-peak is selected from about 1.3 to about 1.9.

In an embodiment, the resolution of pre-peak is from about 1.3, about 1.4, about 1.5, about 1.6, about 1.7, about 1.8, about 1.9.

In an embodiment, the resolution of pre-peak is 1.82.

The present invention provides an example for illustration purpose which should not be considered to limit the scope of the present invention with the described examples.

### EXAMPLES

Process for estimation and/or quantification of pre-peak and main peak of protein mixture comprising CTLA4-IgG1 fusion protein.

### Reagents Details:

a) Sodium phosphate dibasic anhydrous
b) Sodium phosphate monobasic monohydrate
c) Sodium sulphate anhydrous
d) Orthophosphoric acid
e) Sodium chloride
f) Potassium phosphate dibasic anhydrous
g) Potassium phosphate monobasic anhydrous
h) Potassium chloride
i) Milli Q water

### Equipment Details:

a) HPLC system equipped with a pump, an autosampler, a UV detector and a suitable data acquisition system
b) Digital Dry bath
c) Magnetic stirrer
d) pH meter
e) Analytical weighing balance
f) Sonicator
g) Filter assembly
h) 0.2 μm membrane filter

### Example 1

Size exclusion chromatography Column selection: Column of different brands and dimensions were tried which could work for CTLA4-IgG1. Samples were diluted in mobile phase and injected to the column. Percentage (%) purity, USP Resolution and tailing was compared between different columns.

Sample (CTLA4-IgG1 fusion protein) was diluted from 25 mg/ml to 1 mg/ml in mobile phase. 30 μg sample was injected (injection volume 30 μl).

### Chromatographic Conditions:

| | |
|---|---|
| HPLC system: | HPLC system equipped with a pump, an autosampler, a |
| | UV detector and a suitable data acquisition system |
| Columns: | Biosep SEC s2000 & TSKgel G3000swxl. |
| Mobile Phases: | Biosep SEC s2000: |
| | 100mM Sodium phoshphate + 100mM Na2SO4, |
| | pH 6.5 |
| | TSKgel G3000swxl: 100 mM potassium phosphate + |
| | 200 mM KCl, pH 6.5 |
| Mode: | Isocratic |
| Detection: | UV at 215nm |
| Flow Rate: | 0.5 ml/min |
| Injection Volume: | 30 μl |
| Injection Amount: | 30 μg |
| Column Temp.: | 30° C. |
| Sample Temp.: | 4-8° C. |
| Run time: | 60 minutes |
| Needle Wash: | 5% (v/v) Methanol in water |

The experiment is performed by incorporating injection/s of the blank solution followed by injection/s of reference protein standard onto mentioned chromatographic columns. Test sample which is CTLA4-IgG1 is injected onto the columns thereafter.

TABLE 1
Results for CTLA4-IgG reference in different columns

| | | Total Post- | | | |
|---|---|---|---|---|---|
| | Total Pre- | peak | | USP | |
| | peak area | area | | resolution | |
| | percentage | percentage | Percentage | of | USP |
| Column | (%) | (%) | (%) Purity | pre-peak | tailing |
| Biosep SEC | 0.94 | 0.53 | 98.52 | 1.82 | 1.12 |
| s2000 | | | | | |
| TSK gel | 1.29 | Not | 98.71 | 2.03 | 1.13 |
| G3000swxl | | detected | | | |

As shown in table 1, Biosep SEC s2000 has able to detect and quantify the post peak having 0.53% total area, where TSK gel G3000swxl fails to quantify post peak.

### Example 2

Quantification of pre-peak, post-peak and main peak of protein mixture containing CTLA4-IgG1 fusion protein.

Sample (CTLA4-IgG1 fusion protein) was diluted from 25 mg/ml to 1 mg/ml in mobile phase, 30 μg sample was injected (injection volume 30 μl).

### Chromatographic Conditions:

| | |
|---|---|
| HPLC system: | HPLC system equipped with a pump, an autosampler, a |
| | UV detector and a suitable data acquisition system |
| Column: | Biosep SEC s2000 |
| Mobile Phase: | Mobile phase (with column Biosep SEC s2000): 100 |
| | mM Sodium phosphate in combination with 100 |
| | mM Na₂SO₄, pH 6.5 |
| Mode: | Isocratic |
| Detection: | UV at 215 nm |
| Flow Rate: | 0.5 ml/min |
| Injection Volume: | 30 μl |
| Injection Amount: | 30 μg |
| Column Temp.: | 30° C. |
| Sample Temp.: | 4-8° C. |
| Run time: | 60 minutes |
| Needle Wash: | 5% (v/v) Methanol in water |

The experiment is performed by incorporating injection/s of the blank solution followed by injection/s of reference protein standard onto chromatographic column Biosep SEC s2000. Test sample which is CTLA4-IgG1 fusion protein is injected onto Biosep SEC s2000 column thereafter.

TABLE 2
Results for Percentage (%) purity or Main peak area percentage
(%), pre-peak area percentage (%) and post-peak area percentage
(%) in Biosep SEC s2000 column:

| | | | Total Post- | | |
|---|---|---|---|---|---|
| | Total Pre-peak | | peak area | USP | |
| | area percentage | Percentage | percentage | resolution | USP |
| Column | (%) | (%) Purity | (%) | of pre-peak | tailing |
| Biosep SEC | 0.94 | 98.52 | 0.53 | 1.82 | 1.12 |
| s2000 | | | | | |

As shown in Table 2, Biosep SEC s2000 column provides 98.52% purity and 0.94% of total pre-peak area and 0.53% of total post-peak area and 1.82 pre-peak resolution of CTLA4-IgG1 fusion protein.

### Example 3

Comparison of mobile phase for quantification of pre-peak, post-peak and main peak of protein mixture containing CTLA4-IgG1 fusion protein.

For SE HPLC, mobile phase with either sodium or potassium salt can be used. Sodium or potassium salts with different salt concentration were used with Biosep SEC column. Resolution of impurities was compared between different mobile phases.

Sample (CTLA4-IgG1 fusion protein) was diluted from 25 mg/ml to 1 mg/ml in mobile phase. 30 μg sample was injected (injection volume 30 μl).

### Chromatographic Conditions:

| | |
|---|---|
| HPLC system: | HPLC system equipped with a pump, an autosampler, a |
| | UV detector and a suitable data acquisition system |
| Column: | Biosep SEC s2000 |
| Mobile Phase: | Mobile phase (with column Biosep SEC s2000): |
| | a) 100 mM Sodium phosphate in combination with |
| | 100 mM Na2SO4, pH 6.5 |
| | b) 100 mM Sodium phosphate in combination with |
| | 200 mM Na2SO4, pH 6.5 |
| | c) 100 mM Sodium phosphate in combination with |
| | 100 mM NaCl, pH 6.5 |
| | d) 100 mM Sodium phosphate in combination with |
| | 200 mM NaCl, pH 6.5 |
| | e) 100 mM Potassium phosphate in combination with |
| | 200 mM KCl, pH 6.5 |
| Mode: | Isocratic |
| Detection: | UV at 215 nm |
| Flow Rate: | 0.5 ml/min |
| Injection Volume: | 30 μl |
| Injection Amount: | 30 μg |
| Column Temp .: | 30° C. |
| Sample Temp .: | 4-8° C. |
| Run time: | 60 minutes |
| Needle Wash: | 5% (v/v) Methanol in water |

In present example, all the column conditions were kept constant except the mobile phase. Applicant has tried both the above-mentioned mobile phase a) to e) to observe the effect of mobile phase over the quantification of pre-peak, post-peak and main peak of protein mixture containing CTLA4-IgG1 fusion protein.

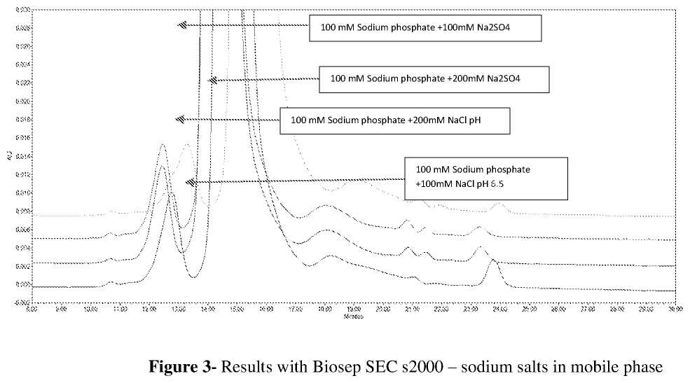

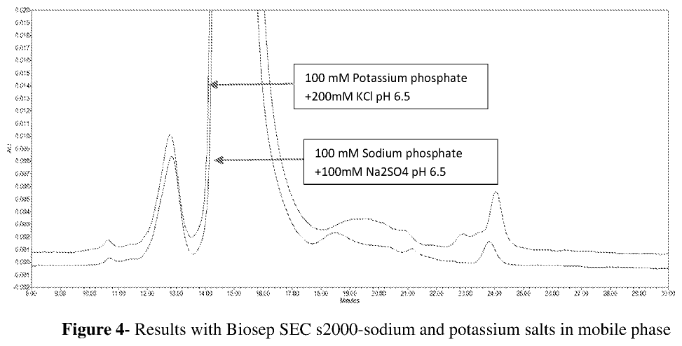

In Biosep SEC s2000 column, Sodium phosphate with 100 mM Na2SO4, 100 mM and 200 mM NaCl gave comparable profile as shown in FIG. 3. Pre-peak broadening was observed with 200 mM Na2SO4. When mobile phase with potassium salt was used with Biosep column, post peak impurity becomes broad (peak shape is distorted) as seen in FIG. 4.

Hence with Biosep SEC s2000 column mobile phase with sodium salts that is 100 mM Sodium phosphate in combination with 100 mM Na2SO4, pH 6.5 showed adequate pre-peak area or area percentage (%) and post peak area or area percentage (%), good purity and sharp resolution of pre-peak, post peak and main peak of protein mixture containing CTLA4-IgG1 fusion protein.

### Example 4

Flow rate optimization study for quantification of pre-peak and main peak of protein mixture containing CTLA4-IgG1 fusion protein:

Two different flow rates 0.5 ml/min and 0.3 ml/min were tried for SE HPLC with TSK gel G3000swxl column.

Experimental details for flow rate optimization:

| Sample | | |
|---|---|---|
| preparation | Chromatographic | |
| details | conditions | Processing method |
| Reference | Column: TSKgel | 0.5 ml/min flow rate |
| Sample | G3000swxl Column | Integration Algorithm: Apex |
| (CTLA4-IgG1) | Temperature: 30° C. | Track Start time: 10.5 min |
| was diluted from | Mode: Isocratic | End time: 21 min Peak width |
| 25 mg/ml to 1 | Run time: 60 min | (sec): 50 |
| mg/ml in mobile | Detection wavelength: | Detection threshold: 7.000e+01 |
| phase. | 215 nm | Suitability parameter: On |
| 30 μg sample | Mobile phase: 100 mM | 0.3 ml/min flow rate |
| was injected | Potassium phoshphate + | Integration Algorithm: Apex |
| (injection | 200 mM KCl, pH 6.5 | Track Start time: 17 min |
| volume 30 μl). | Flow rate 1: 0.5 ml/min | End time: 35 min |
| | Flow rate 2: 0.3 ml/min | Peak width (sec): 50 |
| | | Detection threshold: 8.000e+01 |
| | | Suitability parameter: On |

TABLE 3
Comparative data for different flow rate:

| | Total Pre- | Total Post | | | |
|---|---|---|---|---|---|
| | peak area | peak area | | | |
| Flow rate | percentage | percentage | Percentage | Pre-peak | |
| (ml/min) | (%) | (%) | (%) purity | resolution | USP tailing |
| 0.5 | 1.29 | Not detected | 98.71 | 2.03 | 1.13 |
| 0.3 | 1.05 | Not detected | 98.94 | 2.04 | 1.10 |

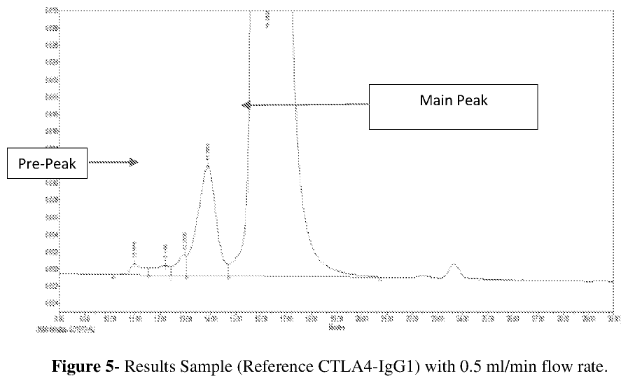

Above table 3 data shows, total pre-peak area percentage (%) was slightly lower with 0.3 ml/min flow rate as compared to 0.5 ml/min. Hence 0.5 ml/min flow rate was finalized. Refer FIGS. 5 & 6. However. TSKGel is failed to detect postpeak in CTLA4-IgG1 column.

1. A method for the separation of CTLA4-IgG1 fusion protein mixture comprising CTLA4-IgG1 fusion protein, pre-peak and post-peak impurity thereof, the process comprises;
a) loading the CTLA4-IgG1 fusion protein mixture onto Size Exclusion High Performance Liquid Chromatography (SE-HPLC) column;
b) separating the protein mixture with suitable mobile phase comprising combination of salts at suitable pH more than pI of the fusion protein; wherein the mobile phase maintains flow rate more than 0.3 mL/min;
c) separating the pre-peak impurity and post-peak impurity from the CTLA4-IgG1 fusion protein of interest; wherein the separation has pre-peak resolution is more than 1.3.

2. The method according to claim 1, wherein the pre-peak, post peak and said CTLA4-IgG1 fusion protein is further quantified at suitable detection absorbance selected from about 214 nm to about 280 nm.

3. The method according to claim 1, wherein the protein mixture is obtained from harvest, partially purified, substantially purified by any other purification methods.

4. The method according to claim 1, wherein the protein mixture is obtained from affinity chromatography, preferably protein A chromatography.

5. The method according to claim 1, wherein the pre-peak impurity is high molecular weight and/or aggregates and post-peak impurity is low molecular weight and/or fragments of CTLA4-IgG1.

6. The method according to claim 1, wherein the mobile phase is selected from sodium phosphate in combination with sodium sulphate, potassium phosphate in combination with potassium chloride, sodium phosphate in combination with potassium chloride, and potassium phosphate in combination with sodium sulphate in suitable concentration selected from about 50 mM to about 150 mM.

7. The method according to claim 6, wherein the mobile phase is selected from sodium phosphate in combination with sodium sulphate in suitable concentration selected from about 80 mM, about 90 mM, about 100 mM, about 110 mM, and about 120 mM.

8. The method according to claim 1, wherein the mobile phase comprises salt selected from sodium sulphate, potassium chloride, in suitable concentration selected from about 50 mM to about 150 mM.

9. The method according to claim 8, wherein the salt concentration is selected from about 80 mM, about 90 mM, about 100 mM, about 110 mM, and about 120 mM.

10. The method according to claim 1, wherein the suitable pH is about 5.5 to about 7.0, preferably about 6.5 to about 6.7.

11. The method according to claim 1, wherein the mobile phase is free of sodium chloride, arginine, acetonitrile, TFA, guanidine hydrochloride, urea and formic acid.

12. The method according to claim 1, wherein the loading of the fusion protein mixture comprises about 30 μg/μl to about 80 μg/μl.

13. The method according to claim 1, wherein the separation performed at flow rate selected from about 0.4 ml/min, about 0.5 ml/min, and about 0.6 ml/min.

14. The method according to claim 1, wherein SE-HPLC comprises size exclusion column having silica matrix, pore size selected from about 140 Å to about 290 Å and dimension selected from 150*4.6 mm, 300*7.8 mm.

15. The method according to claim 14, pore size of the SE-HPLC is 14.5 nm or 145 Å to about 20 nm or 200 Å and dimension 300*7.8 mm.

16. The method according to claim 14, wherein the size exclusion column is BioSep-SEC-S2000, BioSep-SEC-S3000, BioSep-SEC-S4000.

17. The method according to claim 1, wherein the peak tailing is from about 0.7 to 1.15.

18. The method according to claim 1, wherein the pI of CTLA4-IgG1 is less than 6.5.

19. The method according to claim 1, the pre-peak area and post-peak is not merged or interfered with main peak area.

20. The method according to claim 1, the purity of fusion protein of interest or main peak more than 98%.
